(19) United States
(12) Patent Application Publication
(10) Pub. No.: US 2024/0285703 A1
(43) Pub. Date: Aug. 29, 2024

(54) Method For Extracting And Purifying Broad-Spectrum Cannabis Oil From Industry Hemp

(71) Applicant: Zhejiang Shuangzi Intelligent Equipment Co., Ltd., Hangzhou (CN)

(72) Inventors: Zhibiao Zheng, Hangzhou (CN); Hanyu Tian, Hangzhou (CN)

(21) Appl. No.: 18/114,243

(22) Filed: Feb. 25, 2023

(51) Int. Cl. A61K 36/185

(52) U.S. Cl. CPC A61K 36/185; A61K 2236/15; A61K 2236/17; A61K 2236/333; A61K 2236/51; A61K 2236/53; A61K 2236/55

(57) ABSTRACT

A method for extracting broad-spectrum cannabis oil from industry hemp includes the following steps: the hemp leaves, flowers drying, crushing to obtain the material powder, the material powder was extracted by ethanol with concentration value of 60-95%(V/V) to get extract, then do the process of concentration, decarboxylation, using short-range molecular distillation to remove impurities, distillation liquid winterization, pressure filtration to remove wax, filter solution into the column to remove impurities, purification, separation. The purified ethanol chromatographic solution to be concentrated in order to remove alcohol and water product namely the broad-spectrum hemp oil, this method may obtain the product of tetrahydrocannabinol (THC) content can control below 0.1%, the product safety is high, completely satisfies the industrial production.

### FIELD OF THE INVENTION

The invention belongs to the field of plant extraction and purification, in particular to a method for extracting and purifying broad-spectrum cannabis oil from industrial industry hemp.

### BACKGROUND OF THE INVENTION

Industrial industry hemp (Cannabis sativa L.) is a genus of the family Sangaceae with annual erect herbs. The active ingredients of cannabis plants include cannabinoids and non-cannabinoids, in which the number of cannabinoids and active ingredients are relatively. cannabinoids mainly include tetrahydrocannabinol (THC), cannabidiol (CBD), cannabinol (CBN), cannabigerol (CBG), cannabichromene (CBC). Tetrahydrocannabinol (THC), cannabidiol (CBD), cannabinol (CBN), which are the main cannabinoids in the cannabinoids family. Cannabidiol (CBD) is the most content in the canabinoids family, tetrahydrocannabinol (THC) will make the user exited and and hallucinations, therefore, it is highly restricted in the field of application, and conversely, cannabidiol (CBD) is not addictive to hallucinations, not only is oxidation. The difference between cannabidiol (CBD) monomer and broad-spectrum CBD oil, the broad-spectrum oil composition is more extensive, including flavonoids, terpenes, cannabinoids and so on, in which the cannabinoids content is more than 80%, the CBD monomer does not contain flavonoids, terpenes, and the CBD content is more than 99%.

In recent years, scholars have found that broad-spectrum CBD oil treatment effect and industry use is larger than CBD monomer, at present, the European and American market, the use of broad-spectrum CBD oil, than CBD monomer, broad-spectrum CBD oil production, processing has a great potential, the development of broad-spectrum CBD oil production process is of great significance.

The existing technology mainly extracts crude oil with full spectrum oil and broad spectrum oil, but which still contains THC, which affects circulation and limits the use of people.

The existing extraction methods of broad-spectrum CBD oil purification and extraction are static and timed, low efficiency, supercritical CO2 fluid extraction technology has high equipment cost, high operation cost and low efficiency to achieve large scale production.

In the current extraction process, solvent extraction is more common, using hexane, methyl acetate, petroleum ether, dichloromethane, chloroform, methanol and so on. Although the impurity content of the extract is relatively low than that of ethanol, the use of too many organic solvents will be unsafe, destroy the environment, and the residue will be difficult to remove.

### SUMMARY OF THE INVENTION

The invention provides a method for extracting and purifying broad-spectrum CBD oil from industry hemp, which is low cost, can achieve continuous extraction with only one solvent of ethanol, extracting broad-spectrum CBD oil, and the THC content less than 0.1%.

A method for extracting and purifying cannabidiol (CBD) from industry hemp is as follows:

Step 1: Dry the hemp flowers and leaves and place the crushed leaves for use.

Step 2: The material enters the continuous countercurrent extraction unit or the high efficiency centrifugal extraction unit through the conveyer.

Step 3: Decarboxylate the extract at 130˜150° C.

Step 4: Put the decarboxylate into the short-path molecular distillation and collect the crude oil with different boiling points.

Step 5: Winterize the crude oil from the distillation, with a winterization temperature of −60˜−20° C.

Step 6: Dilute the miscellaneous, refining and separating from the column with ethanol solution. The target product was collected by gradient elution of ethanol

### Solution

Step 7: Combine the eluted target product and use low-temperature concentrate to obtain the broad-spectrum cannabinoid oil from the ointment.

The invention has the advantages of:

(1) The invention adopts two extraction methods, one is a countercurrent extraction unit and the other is a high efficiency centrifugal extraction unit. countercurrent extraction is suitable for large-scale production, continuous incoming and outgoing materials, high efficiency and solvent save. The high efficiency centrifugal extraction unit is suitable for scientific research units and family workshops, the extraction quantity is relatively small, the timed type extraction, the advantage extraction time is short, only needs 30˜60 min, the solvent consumption is less than 3˜5 material solvent.

(2) The invention adopts filler adsorption separation, the THC separation effect is good, the pure broad-spectrum CBD oil can be obtained, the finished product has almost no detection of THC, and other active components are retained.

(3) There are few types of solvents used, and only one solvent, ethanol, is easy to manage safely and reduces product residue risk.

### BRIEF DESCRIPTION OF THE DRAWINGS

FIG. 1 is the high performance liquid chromatography (HPLC) of the raw material extract of the invention.

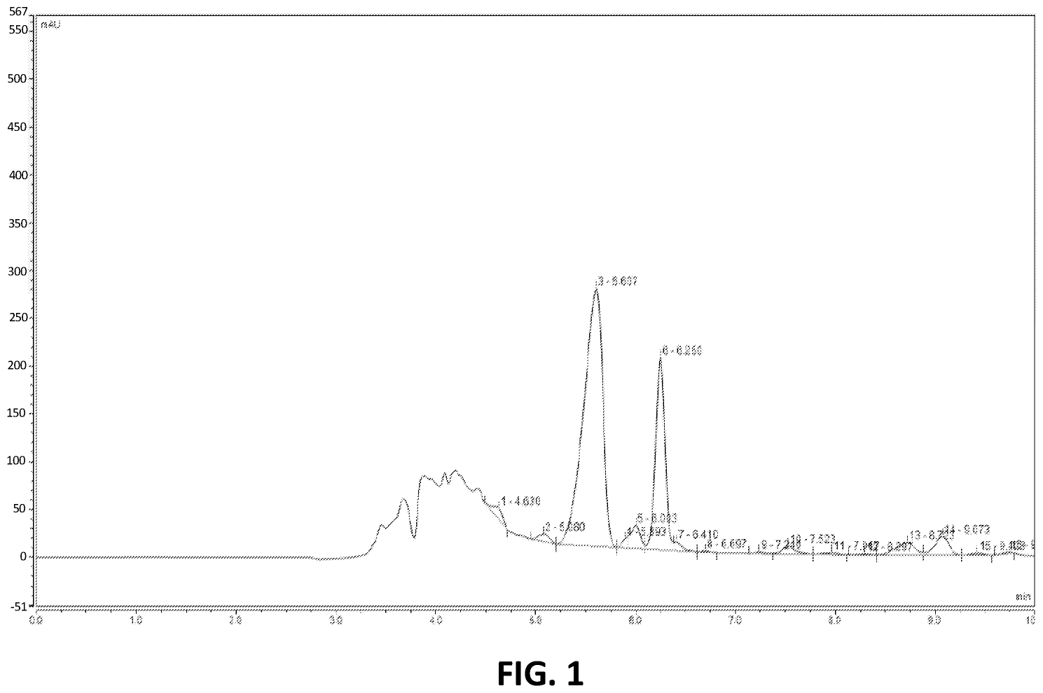

FIG. 2 is a high performance liquid chromatography (HPLC) of finished broad-spectrum CBD oil (CBD) prepared from implementation 1 of the invention.

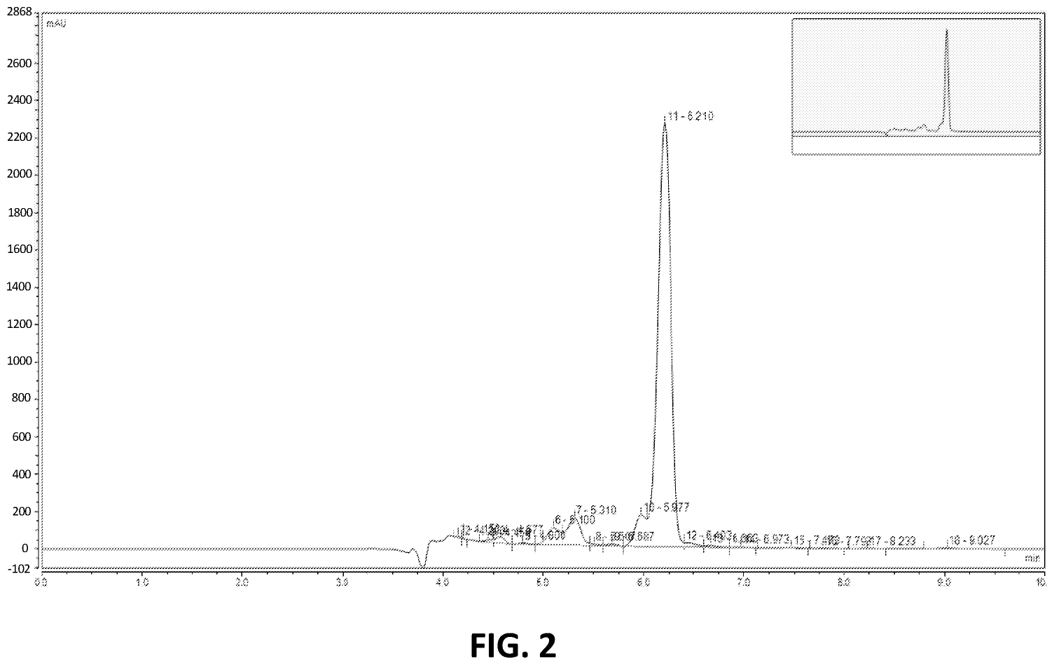

FIG. 3 is a high performance liquid chromatography (HPLC) of finished broad-spectrum CBD oil (CBD) prepared from implementation 2 of the invention.

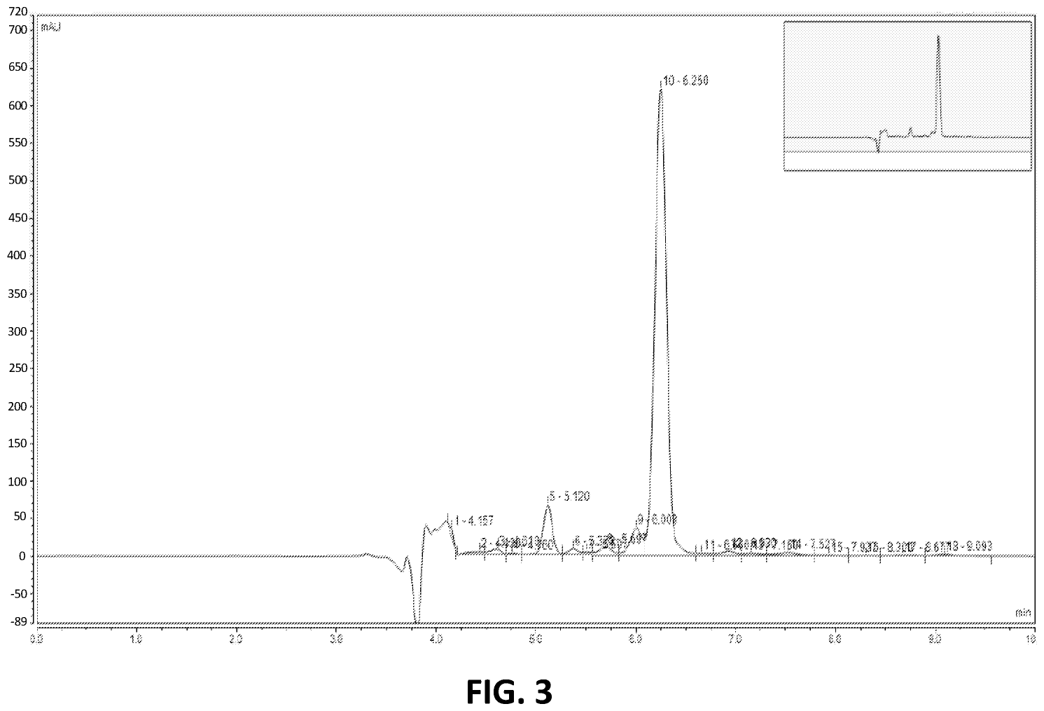

### DETAILED DESCRIPTION OF THE PREFERRED EMBODIMENTS

The patent of the invention is described in detail in conjunction with an example.

Implementation 1: The present example provides the following steps for extracting and purifying broad-spectrum CBD oil from industry hemp:

Cut the industry hemp leaves and flowers, dry until water content≤5%, crush the material to 60 mesh to get the clean raw material to be used.

weighing 60 kg of hemp powder (clean raw material), according to the feed rate of 15 kg/h continuous feeding, solvent using 8 times 95 v/v of ethanol solution, ethanol solution according to 120 l/h continuous feed, extraction temperature is room temperature, extraction time is 60 minutes, extraction completed collection of the extract to obtain the target product.

The extract was concentrated in a vacuum low-temperature concentrator at a concentration temperature of 50˜70° C. to get extractum, and the extractum was continuously heated to 140° C. for 90 min to get the aimed product.

The crude oil was obtained by distilling the decarboxylate solution for short-path distillation at 120-280° C.

The short-path distilled solution was diluted with 5 times by 85 V/V ethanol, and the diluent was cooled to −40° C. for 8 h, and the impurities such as wax were removed by freezing pressure filtration.

The filter fluid was decolored by organic bentonite column, then washed with double column volume of 80 V/V ethanol and then regenerated with 95 V/V ethanol. Among them, the perforating liquid, the eluant as a useful component, the material after decolorization into the reverse C18 dynamic high pressure column, the upper sample volume of ½ column volume, using 95V/V equivalent elution, HPLC on-line detection, collection of useful components.

A broad spectrum CBD oil was obtained by vacuum concentrating the ethanol solution of the reverse chromatographic column to be extractum.

### Implementation 2:

Cut the hemp leaves and flowers, dry until water content≤5%, crush the material to 60 mesh to get the clean raw material to be used.

Weighing the 10 kg hemp powder, the weighted material was added to the high efficiency centrifugal extraction unit, the solvent was 4 times 95V/V ethanol solution, the total amount was added 40 L ethanol, the extraction temperature was room temperature, the extraction time was 30 minutes, the rotational speed was 400 r/min, and the extraction was completed to collect the extract solution and obtain the target product.

The extract was concentrated in a vacuum concentrator at a temperature of 50˜70° C. to get extractum, and the extractum was heated to 150° C. for 60 min.

The crude oil was obtained by short-path distillation of the decarboxylate solution at 120-280° C.

The short-path distilled solution was diluted with 5 times 85 V/V ethanol, the dilution solution was cooled to be −40° C., frozen for 8 h, and the impurities such as wax were removed by freezing pressure filtration.

Filter liquid load into decolorate column DM11, load sample: load 5 times column volume material, load sample finish; Flush: use 75V/V ethanol to flush column volume, totally flush 2 times column volume; Clean wash: use 6 times column volume 95V/V ethanol to clean column. Among them, perforation liquid, flushing liquid is useful. The DM11 decolorization liquid load sample AB-8 separation column, load sample: load 7 times column volume material, upper sample finish; Flush: use 70V/V ethanol to flush the column, totally flush 2 times column volume; Clean wash; and then use 5 times column volume 95V/V ethanol to clean the column. Among them, perforation liquid and flushing liquid are useful substances.

The AB-8 as one kind of separations is entered into the evaporator and the material is concentrated to the extractum to obtain a broad spectrum CBD oil.

Table 1. The extraction rate, CBD content and THC detection content of the two different methods were compared.

TABLE 1

| Name of | Extraction | CBD content | THC content | |
|---|---|---|---|---|
| product | rate (%) | (wt %) | (wt %) | Chromatography |
| Product 1 | 98.4% | 83.7 | No detection | FIG. II |
| Product 2 | 98.1% | 84.6 | No detection | FIG. III |

From the results of the two tests, cannabinoids are more than 80%, The THC content is less than 0.1%, and the characteristics of the examples are not fully expressed one by one, so the above methods can be combined with each other.

What is claimed is:

1. A method for extracting and purifying broad-spectrum cannabis oil from industry hemp, comprising steps of:
(1) Drying and crushing hemp leaves and flowers to get extracted clean raw material;
(2) Extracting a first substance from the clean raw material with ethanol of 60-95%(v/v);
(3) Concentrating and decarboxylation of the first substance;
(4) Distilling the decarboxylate solution in step (3) to obtain an oil that is separated by different boiling points;
(5) Dewaxing the oil in step (4) through pressure filtration at low temperature;
(6) Purifying the filtered solution in step (5) by chromatography; and
(7) Obtaining a broad spectrum oil by concentrating the purified liquid in step (6).

2. A method for extracting and purifying broad-spectrum cannabis oil in claim 1, wherein the hemp leaves are dried and crushed under the following condition: the drying temperature is ≤150° C., the water content is ≤5%, and the number of crushed mesh is from 20 mesh to 80 mesh.

3. A method for extracting and purifying broad-spectrum cannabis oil in claim 1, wherein step (2) is a countercurrent extraction carried out by a countercurrent extraction equipment, and the ethanol is extracted at room temperature with ethanol 4˜20 times more than the clean raw material, and the extraction time is 30 minutes to 120 minutes.

4. A method for extracting and purifying broad-spectrum cannabis oil in claim 3, wherein step (2) is a high efficiency centrifugal extraction with ethanol 3 to 5 times more than the clean raw material at room temperature, for 30 to 60 minutes.

5. A method for extracting and purifying broad-spectrum cannabis oil in claim 1, wherein in step (3), the decarboxylation process in step (5) is under the temperature of 130° C. to 150° C. for 1 to 2 hours.

6. A method for extracting and purifying broad-spectrum cannabis oil in claim 1, wherein in step (4), a distillation temperature is 160° C. to 280° C.

7. A method for extracting and purifying broad-spectrum cannabis oil in claim 1, wherein in step (5), the temperature is −60° C. to −30° C., for 5 to 12 hours.

8. A method for extracting and purifying broad-spectrum cannabis oil in claim 1, wherein in step (6), the chromatography's column gradient elution is ethanol, conducted as the following steps: removing impurities with 30 to 70%(V/V), washing with 50 to 90%(V/V), and the regenerating with 90 to 95%(V/V) or purified water, and the fillers used for chromatography include one or more macroporous resins as NM200, SD300, DM11, AB-8, HPD-703, LS-7, and organic bentonite, and reverse chromatographic filler silica gel C18.

9. A method for extracting and purifying broad-spectrum cannabis oil in claim 1, wherein in step (7), a vacuum concentrator is used to heat the purified product in step (6) to become completely oily.
